(19) United States
(12) Patent Application Publication
(10) Pub. No.: US 2025/0041611 A1
(43) Pub. Date: Feb. 6, 2025

(54) BUS DISTRIBUTED STIMULATION AND MEASUREMENT SYSTEM

(71) Applicants: NEURINNOV, MONTPELLIER (FR); INRIA - INSTITUT NATIONAL DE RECHERCHE EN INFORMATIQUE ET EN AUTOMATIQUE, Le Chesnay (FR); UNIVERSITÉ DE MONTPELLIER, Montpellier (FR)

(72) Inventors: David ANDREU, Les Aires (FR); Milan DEMARCQ, Les Aires (FR); David GUIRAUD, Les Aires (FR)

(73) Assignees: NEURINNOV, MONTPELLIER (FR); INRIA - INSTITUT NATIONAL DE RECHERCHE EN INFORMATIQUE ET EN AUTOMATIQUE, Le Chesnay (FR); UNIVERSITÉ DE MONTPELLIER, Montpellier (FR)

(21) Appl. No.: 18/716,360

(22) PCT Filed: Dec. 8, 2022
(86) PCT No.: PCT/FR2022/052280

§ 371 Date: Jun. 4, 2024

(30) Foreign Application Priority Data
Dec. 8, 2021 (FR) FR2113118

(51) Int. Cl. A61N 1/372; A61N 1/36

(52) U.S. Cl. CPC A61N 1/37288; A61N 1/3614

(57) ABSTRACT

A stimulation system designed to be implanted in a person and including several distributed elements connected by a wire bus comprising an energy wire, a data wire and a reference wire.

### FIELD

The present invention relates to implanted stimulation systems, in particular the architectures of implanted communication networks ensuring the interconnection of active implantable medical devices whose functions are centered on stimulation, measurement and control.

### BACKGROUND

The improvement in the quality of life of people with loss of motor loss or sensory abilities—as a result of trauma, medullary or cervical for example, or following another assignment, multiple sclerosis, for example—is a challenge. The functional electrical stimulation technologies, intended to cause, inhibit, or modulate a nervous message in order to restore the motor or sensory functions have been widely explored. But many difficulties remain in order to obtain high-performance implanted stimulation systems.

In particular, an implanted stimulation system may require the networking of several units: control, stimulation, possibly measurement. These units must exchange information with one another. These units must also be supplied with energy. Finally, the constraints of implantation in the body of a patient—or of placement in a natural orifice-lead to limiting the bulk of the units and impose a control of the charges and electrical currents exchanged between the units and which must not leak into the intra-body medium of the person equipped with the implanted stimulation system.

For example, the US patent application US 2008/0061630 discloses the use of several implants connected individually to a central unit. However, such a type of connection does not allow to verify the presence of a potential drift. The US patent application US 2008/0061630 system therefore does not allow reliable and robust measurement between the various implants.

An object of the invention is therefore to propose a stimulation system designed to be implanted partially or entirely and allowing to transfer energy and information between several units, while avoiding undesirable electrical currents.

### SUMMARY

The invention therefore relates to a stimulation system adapted to be implanted in a person comprising:
distributed elements comprising:
   a controller comprising an energy emission module and a data emission/reception module; and
   at least one remote unit comprising at least one energy recovery module and at least one data emission/reception module;
a wired bus connecting all the distributed elements, said bus comprising:
   a common reference wire connecting all the distributed elements;
   an energy wire connected via a coupling capacitor to the energy emission module of the controller; and connected directly to the energy recovery modules of the at least one remote unit; and
   a data wire connected via a coupling capacitor to each of the data emission/reception modules of all the distributed elements.

In one embodiment, the at least one remote unit are chosen from stimulation units, measurement units and mixed stimulation and measurement units. In particular, the stimulation system comprises at least two stimulation units and at least one measurement unit.

In one embodiment, at least one remote unit further comprises at least one electrode configured to be in contact with the intra-body medium, in particular for measuring an electrophysiological signal.

In one embodiment, each energy recovery module is assisted by a load balancing module, for example comprising a buffer capacitor.

In one embodiment, the wired bus comprises a spare wire, in particular to:
Transmit event-related data; and/or
Be substituted with the common reference wire, the energy wire or the data wire when one of these wires fails.

In one embodiment, each of the at least one remote unit comprises a switch normally closed between the common reference wire and the spare wire.

The invention also relates to a method for measuring physiological signals by a stimulation system presented above comprising:
connecting to the common reference wire an electrode of the remote unit in contact with the intra-body medium; and
measuring by the controller of a voltage between the common reference wire and a IC medium contact of the controller.

The invention also relates to a method for measuring the contact impedance of an electrode of a stimulation system presented above comprising:
connecting to the common reference wire an electrode of a remote unit in contact with the intra-body medium;
injecting by an intra-body medium contact of the controller a current in the intra-body medium; and
measuring by the controller the voltage between the common reference wire and the IC medium contact of the controller.

The invention also relates to a method for detecting defects of a stimulation system presented above comprising:
injecting by the controller a current in the energy wire or the data wire or the spare wire; and
measuring by the controller the voltage between the common reference wire and the wire in which the current has been injected.

### Definitions

In the present invention, the terms below are defined as follows:
“Direct connection” concerns bringing two electrical circuits into contact without any coupling capacitor.
“Connection via a coupling capacitor” relates to bringing two electrical circuits into contact by means of a coupling capacitor in series, the purpose of which is here to limit the injections of charge in the implanted device and into the intra-body medium.
“IC medium contact” relates to an electrical contact of a distributed controller-controller unit or remote unit—with the immediately adjacent body intra-body medium.
“Normally closed switch” is a controlled switch which is passing-closed—in the absence of a control voltage. Such a switch placed in a remote unit is therefore on when the remote unit is not active-supplied with energy. The normally closed switches may be switches based on Depletion-Mode MOSFETs, J-FETS-based mountings which are Depletion Mode Devices or micro-relays for example.
“Common reference” relates to an electric potential which is common to all the distributed elements-controller or remote unit. This potential is shared by the common reference wire. Each distributed element therefore contains a point whose electrical potential is the same as in the other distributed elements.

### BRIEF DESCRIPTION OF THE DRAWINGS

FIG. 1 shows a stimulation system comprising a controller, three remote units-two stimulation units and one measurement unit—each comprising an electrode.

FIG. 2 schematically represents a controller.

FIG. 3 schematically represents a remote unit.

FIG. 4 schematically represents the configuration of the stimulation system for the method for measuring physiological signals.

FIG. 5 schematically represents the configuration of the stimulation system for the method for measuring the contact impedance of an electrode.

FIG. 6 schematically represents the configuration of the stimulation system for the method for detecting defects of a stimulation system.

FIG. 7 schematically represents an energy recovery module of a remote unit of the stimulation system.

FIG. 8 schematically represents a load balancing module.

In all the figures, the dotted lines represent control functions of one element on another; the arrows indicate the direction of transmission of the signal: control, data or energy.

### DETAILED DESCRIPTION

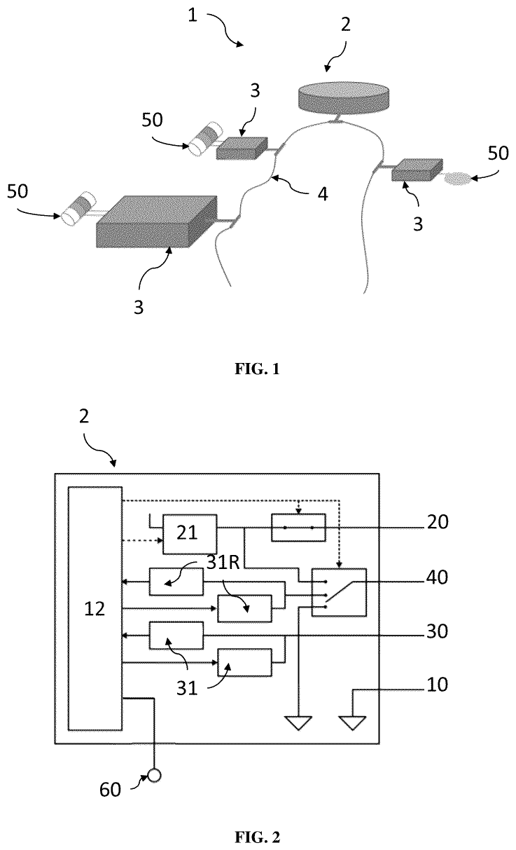

The present invention relates to a stimulation system 1 suitable for being implanted in a person and illustrated in FIG. 1. This system comprises distributed elements and a wired bus 4 connecting all the distributed elements. Each distributed element is connected to all of the distributed elements.

The distributed elements comprise a controller 2, illustrated in FIG. 2. On the one hand, the controller 2 has the function of supplying the distributed elements with energy. For this purpose, the controller 2 comprises an energy emission module 21. The controller 2 may store energy or simply redistribute it. In an implanted configuration, the controller 2 can receive energy by a transcutaneous inductive link and store it in a battery and/or distribute it to the others distributed elements. On the other hand, the controller 2 has the function of controlling the entire system by managing the emission and reception of data between the distributed elements. For this purpose, the controller comprises a data emission/reception module 31.

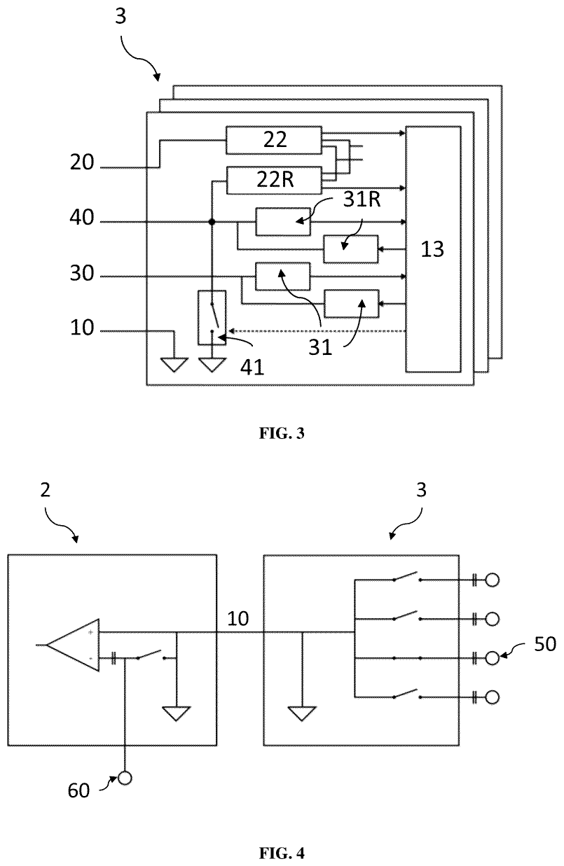

The distributed elements also comprise at least one remote unit 3, preferably several remote units 3, illustrated in FIG. 3, on the one hand, each remote unit s receives controller energy 2. For this purpose, the remote unit(s) 3 comprise an energy recovery module 22. This recovered energy is then consumed as it progresses and/or stored. The storage can be very limited in the capacitors of the energy recovery module, in order to overcome failures due to micro cut-offs. On the other hand, the remote unit(s) 3 are caused to receive and send data to the controller 2. For this, the remote units comprise at least one data emission/reception module 31.

The modules for emitting/receiving the data 31 of the controller 2 and of the remote unit(s) 3 may be identical.

The wired bus 4 connecting all the distributed elements comprises three wires.

First, a common reference wire 10 connects all the distributed elements. This common reference wire 10 allows all the elements distributed to operate with a same potential reference and ensure that the potentials of the distributed elements do not derive from one another, which would lead to discharge currents between the elements distributed via the intra-body medium. However, such currents must be avoided. Thus, the invention is advantageous compared to systems in which several implants are individually connected to a central unit. Indeed, these known systems involve the presence of a reference wire for each implant and not the presence of a single common reference wire 10 for all of the remote units 3 as is the case in the invention. In each of the distributed units, the common reference wire 10 is either unconnected or connected to an IC medium contact of the distributed unit.

In addition, the central unit of the known systems has a fixed architecture limited by the number of connections integrated during the manufacture of the unit. Thus, the known systems cannot be extended and the number of remote units is limited to the number of connections present in the central unit. Thanks to the system of the invention, it is possible to add as many remote units as necessary on the bus.

Secondly, an energy wire 20 connects all the distributed elements. However, this energy wire 20 is connected via a coupling capacitor to the energy emission module 21 of the controller 2. Thus, this capacitive coupling ensures that the only energy source of the system—the controller 2—injects, to the remote unit(s) 3, a current whose DC component is necessarily zero. Indeed, no voltage or current generation system can guarantee perfect balancing of the currents and therefore of the injected charges. The capacitive coupling solves this problem by limiting the charge injections and therefore by preventing the accumulations of electrical charges and the discharge currents in the device and in the intra-body IC medium. Thus, the coupling capacitor ensures that a direct current cannot be transmitted on the wired bus 4 and therefore to the IC medium in the event of failure of the insulation of the energy wire 20. Some known systems include a capacitor mounted in parallel. However, this capacitor simply allows to create an inductive storage chopper. This known connection therefore does not, in contrast to the invention, prevent the accumulations of electrical charges.

At the remote unit(s) 3, the energy wire 20 is connected directly to the energy recovery modules 22. The connection is here direct, but contributes to the same effect: the DC component of the currents entering the remote unit(s) 3 is zero, which prevents the accumulations of electrical charges and the discharge currents in the intra-body medium.

The transmission of the energy from the controller 2 to the distributed unit(s) is done by means of a differential voltage between the energy wire 20 and the common reference wire 10. In order to comply with the constraints of currents having zero DC component, the signal carried by the energy wire 20 is at zero average, but it can take any form adapted to an energy transfer. This signal varies between a maximum positive voltage V+ and a minimum negative voltage V−.

In one embodiment, the energy is transmitted in the form of a symmetrical signal with zero mean current, in the form of a three-state signal. For example, the three states may be associated with voltages of +10 (V+), 0 and −10 (V−) Volts, for a peak amplitude at a peak of 20 V, in alternating square slots—of the same duration in order to ensure the zero mean. The voltage generator may be limited in current to avoid a short-circuit situation at start-up so that the nominal voltages are not necessarily reached instantaneously. The signal may be of low frequency—of an order of magnitude of 10 kHz, typically between 1 kHz and 100 kHz—or of high frequency—of an order of magnitude of 1 MHZ, typically between 100 kHz and 10 MHz. The advantage of the low frequencies is not to pose a problem of electromagnetic compatibility. The advantage of the high frequencies is to allow a smaller dimensioning of the coupling capacitors (and therefore a smaller size) of the system.

The emission of the energy by the controller 2 can be done by an analog multiplexer stage driven by a digital element 12 allowing to produce the three-state signal. Preferably, the energy emission module 21 is based on current sources, thus allowing precise control of the maximum deliverable current and therefore of the maximum energy sent on the bus. For the emission of a three-state signal, the controller 2 has two symmetrical voltages used by the energy emission module 21. In this implementation based on current sources, the voltages are necessarily limited.

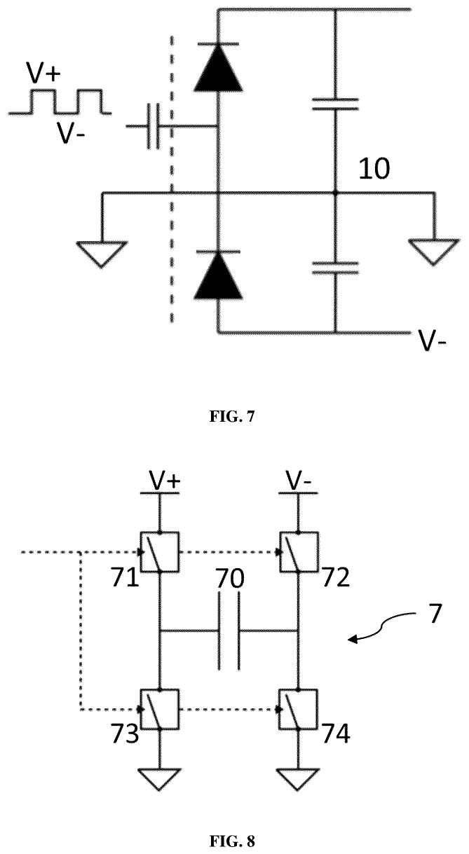

The recovery of the energy by the remote unit(s) 3 is carried out by an energy recovery module 22, for example of the full-wave rectifier type, as illustrated in FIG. 7. In one embodiment, the capacitors used in the rectifier assembly are dimensioned so as to allow temporary energy storage allowing to ensure the operation of the remote unit 3 in the event of a micro cut-offs, performing actions of security of the remote unit 3 or of emergency communication before total extinction of the remote unit 3. In a particular embodiment, the midpoint of the full-wave rectifier is connected to the common reference wire 10. In a particular embodiment, the diodes of the rectifier circuit are Schottky diodes.

Finally, in order to further limit the appearance of imbalances of charges, each energy recovery module 22 may be secondary by a load balancing module 7. Indeed, if the current recovered by a remote unit 3 is consumed in an unbalanced-asymmetrical-on the circuit placed between the common reference wire and the voltage V+ on the one hand and on the circuit placed between the common reference wire and the voltage V-on the other hand, a charge accumulation can occur in the remote unit 3 inducing a drift of the rectified voltages available for the remote units which would then no longer be supplied correctly. To avoid this, a module illustrated in FIG. 8 may be employed, to place a buffer capacitor 70 in an H-bridge This buffer capacitor 70 is connected by controlled switches 71/72/73/74 to the voltages V+ and V-obtained by the energy recovery module and to the common reference wire. When the energy signal is at the value V+, the controlled switches 71 and 74 are passing while the controlled switches 72 and 73 are open; conversely when the energy signal is at the value V−. Thus, the buffer capacitor 70 will accumulate charges in the polarity consuming the least energy—the load of which is of higher impedance—and restore them in the more energy-consuming polarity. the load of which is of lower impedance.

Third, a data wire 30 connects all the distributed elements. The connection of the data wire 30 to the data emission/reception modules 31 of the distributed elements is done via coupling capacitors. The system according to the invention therefore allows to use a common reference wire 10 without capacitive coupling, thus allowing to place a true reference common to all the remote units 3 while having a capacitive coupling on the energy wire 20 and data wire 30. The data transmitted on the data wire 30 can adopt all the usual modes of data transmission, provided that zero mean signals compatible with the capacitive couplings in place are respected and to avoid charge accumulations.

In one embodiment, the data are transmitted using three-state coding of zero mean voltage pseudo-Manchester type—the zero-voltage state corresponding to the common reference wire 10—in which each bit is coded as a succession of two pulses of opposite direction—a pair of pulses—the order of the two successive pulses indicating the value of the transmitted bit.

The transmission of the data is bidirectional: each distributed unit can emit data and receive data.

For each distributed unit, the emission of the data is carried out by an analog electrical circuit controlled by a digital circuit 13: analog multiplexer, switches, etc. Thus, in the case of a pseudo-Manchester type code, each digital datum is converted into a succession of analog pulse pairs on the data wire 30.

For each distributed unit, the reception of the data is carried out by an electrical conversion circuit, for example an analog/digital converter. In the case of a pseudo-Manchester type code, the analog signal has three states, the electrical conversion circuit transforms each succession of pairs of pulses into a succession of bits available on a digital output.

In the case of a pseudo-Manchester type code, the successions of bits can serve as a support for each remote unit in order to reconstruct a clock signal. This avoids resorting to different clocks for each remote unit 3 leading to drifts and synchronization problems.

According to one embodiment, the remote units 3 are chosen from stimulation units, measurement units and mixed stimulation and measurement units. The stimulation units are configured to receive a setpoint from the controller 2 and to apply a signal in the intra-body medium in order to induce, for example, a movement or a sensation. This signal may be electrical, optical or magnetic. The measurement units are configured to measure a physiological signal or a parameter of the intra-body medium, and then send it to the controller. The physiological signals may be electrical-electrocardiographic (ECG), electromyographic (EMG), electroencephalographic (EEG), electroneurographic (ENG), electrocorticographic (ECoG), etc. or other signals that do not require electrical contact with the intracorporeal medium (temperature, acceleration, etc.) or also biochemical measurements: glucose, dissolved oxygen, etc. According to a particular case of this embodiment illustrated in FIG. 1, the stimulation system comprises at least two electrical stimulation units and at least one measurement unit. For example, the multi-site stimulation of an upper limb comprises a controller 2, a first remote unit 3 for electrical stimulation of the forearm, a second remote unit 3 for electrically stimulating the top of the arm and a remote unit 3 for EMG measurement implanted in order to detect the intention—the command—of the patient. This configuration therefore allows a patient who has lost the use of his arm to activate it using an uninjured muscle which gives an instruction via the EMG measurement and the controller 2 to the electrical stimulation units.

According to one embodiment, at least one remote unit 3 further comprises at least one electrode 50 configured to be in contact with the intra-body medium, as illustrated in FIG. 1. Various electrodes 50 may be implemented. A remote unit 3 may comprise an electrode 50 configured to acquire an electrophysiological signal, in particular an EMG, EEG, ENG, ECOG or ECG signal. A remote unit 3 may also comprise an electrode 50 configured to inject a current into the target structure and induce a movement. For example, the epimysial electrodes, placed on the surface of a muscle, intramuscularly, placed inside a muscle are adapted. Moreover, the neural electrodes can be used. The neural electrode may be chosen from Flat Implanted Neural Electrode (FINE), intended to be placed on the nerve; the intra- or inter-fascicular electrodes, intended to be placed inside the nerve; or the cuff electrodes intended to be placed around the nerve. Finally, a remote unit 3 may comprise an electrode 50, the only role of which is to establish an electrical link with the body-environment intra-body IC medium—in other words to connect the remote unit 3 to the potential of the body in which it is implant. The controller 2 may also comprise an electrode 60, the only role of which is to establish an electrical link with the intra-body IC medium.

According to one embodiment, the wired bus 4 further comprises a spare wire 40. The spare wire 40 has the purpose of providing reactivity and robustness to the stimulation system 1. The spare wire 40 can be used in two modes. In normal operating mode, the spare wire 40 can be used to transmit data of event nature from the remote units 3 to the controller 2. This operation allows to transmit data not provided by the protocol in force on the data wire and allows to increase the reactivity of the stimulation system 1 by reducing the time between the detection of an event by a unit remote 3 and the notification of this event to the controller 2, and therefore the time between the detection of an event and the possible action that it drives. It is thus possible to reduce the frequency of the polling periods of data by the controller 2—without loss of reactivity and therefore to reduce the energy consumption due to communications.

In a failure mode, corresponding to the impossibility for the controller 2 to exchange data or energy with a remote unit 3, the spare wire 40 may be substituted for the faulty wire. The transmission of data of event nature is then lost, but the operational continuity of the stimulation system is ensured.

In the controller 2—illustrated in FIG. 2—the spare wire 40 is connected either to the energy emission module 21 of the controller via a coupling capacitor, or to a data emission/reception spare module 31R via a coupling capacitor, or to the common reference. The reconfiguration of the spare wire 40 is carried out by a set of analog switches and multiplexers capable of direct the various signals generated (energy, data) to the controller 2 and the remote unit(s) 3.

In each of the remote unit(s) 3—illustrated in FIG. 3—the spare wire 40 is connected directly to an energy recovery spare module 22R and to a data emission/reception spare module 31R via a capacitor coupling.

In the normal mode, the spare wire 40 is connected via a coupling capacitor to data emission/reception spare modules 31R of all the distributed elements. These data emission/reception spare modules 31R may be identical to the data emission/reception modules 31 described above. The control of the emission/reception spare module 31R is preferably carried out by a dedicated digital circuit, therefore different from the digital control circuit of the emission/reception module, which allows to manage simultaneously two data exchange protocols on the data wire 30 and the spare wire 40.

Upon detection by the controller 2 of a failure on the data wire 30, the connection of the spare wire 40 in the controller 2 is reconfigured to the data emission/reception modules 31 or alternatively, the control of the data emission/reception spare modules 31R supports the main data exchange protocol.

Upon detection by the controller 2 of a failure on the energy wire 20, the connection of the spare wire 40 is reconfigured to the energy emission module of the controller 21 and to the energy recovery spare modules 22R of the remote unit(s) 3. These energy recovery spare modules 22R may be of the same design as the energy recovery modules 22 described above, but they are well distinct, so that it is possible to recover energy on the energy wire 20 or on the spare wire 40 independently and automatically.

According to one embodiment, the spare wire 40 is connected in the remote unit(s) 3 to the common reference via a normally closed switch 41 and the spare wire 40 can be connected in the controller 2 to the common reference. When the stimulation system 1 is powered on, these switches are in the closed position. As a result, the controller 2 can check with the common reference wire 10 and the spare wire 40 if a fault exists, for example by one measurement of impedance between the two wires or by a data emission protocol allowing to deduce whether the spare and/or reference wires are operating or not. In the event of a failure, the controller 2 can then reconfigure the connection of the spare wire 40 to the common reference and keep the switches closed in the remote units 3. The spare wire 40 then replaces the common reference wire 10 allowing to ensure the operational continuity of the stimulation system 1.

The invention also relates to a method for measuring physiological signals with the stimulation system 1 described above, in which a remote unit 3 comprises an electrode 50 in contact with the intra-body medium. The measurement configuration of this method is illustrated in FIG. 4. In this method, the controller 2 measures the signal between the common reference wire 10—connected to the intra-body medium at the remote unit 3—and the IC medium contact 60 of the controller which here plays the role of measurement reference since it is remote from the measurement point of the electrode 50. This analog signal is a direct measurement—without coding by a digital block, transmission on the data wire and then decoding by a digital block—of an electrophysiological signal at a remote unit 3. In this method for measuring physiological signals, it is necessary for the common reference wire 10 and the IC medium contact 60 of the controller to be separated by an open switch.

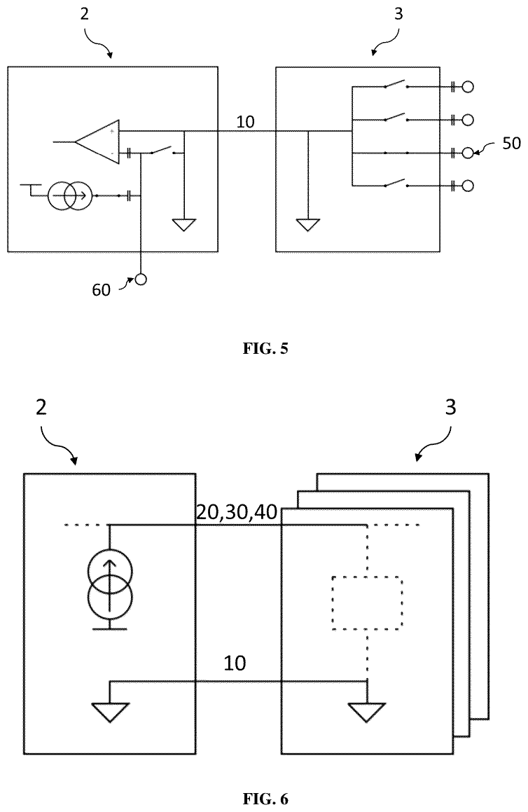

The invention also relates to a method for measuring the contact impedance of an electrode 50 of the stimulation system 1 described above. This method is implemented during the powering on or during the operation of the stimulation system 1. The measurement configuration of this method is illustrated in FIG. 5. In this method, said electrode 50 in contact with the intra-body medium is connected to the common reference wire 10. The controller 2 then injects a current, preferably a low current lower than the natural excitation threshold of the neural or muscle fibers modeled by an ideal source of current-by its IC medium contact 60 in the intra-body medium and measures the signal between the common reference wire 10—connected to the intra-body medium at the remote unit 3—and the IC medium contact 60 of the controller. As the current return path passes through the intra-body medium, the electrode 50 and the common reference wire 10, The voltage necessary to impose the current on this path allows to evaluate the equivalent impedance of the circuit. Since the common reference wire 10 is not open, since the impedance of the intra-body medium is very low and the impedance of the IC medium contact 60 of the controller is also low—and common in any case—the equivalent impedance on this path corresponds essentially to the impedance of contact of the electrode 50 of the stimulation system 1. This method allows to evaluate the electrical characteristics of each of the contacts in the body intra-body by electrode 50 of the stimulation system 1 and to evaluate the failures or the drifts.

The invention also relates to a method for detecting the defect of a stimulation system 1 described above and comprising a spare wire 40. The measurement configuration of this method is illustrated in FIG. 6. In this method, the controller 2 does not emit energy to the remote units 3. The controller 2 injects a current, preferably a low current lower than the natural excitation threshold of the neural or muscular fibers-modeled by an ideal source of current—in the energy wire 20 or the data wire 30 and measures the voltage between the common reference wire 10 and the wire into which the current was injected. If abnormal short circuits are present in a remote unit 3, the current setpoint will not be reached due to the leakage of current by the common reference wire 10 via a low impedance circuit represented by the dotted component, which will allow the controller 2 to detect the failure of the wire into which the current has been injected and reconfiguring the spare wire 40 to replace it with the failed wire. In the absence of a normally closed switch 41 between the spare wire 40 and the common reference in the remote unit(s), this method can also be applied to the spare wire 40. Preferably, the voltage measurement is carried out while the remote units are off, not powered.

The invention also relates to a method for detecting the defect of a stimulation system 1 described above and comprising a spare wire 40 and a normally closed switch 41 between the spare wire 40 and the common reference in the spare wire 40. In this method, the controller 2 injects a current into the spare wire 40 and measures the voltage between the common reference wire 10 and the spare wire 40. In the absence of a failure, the spare wire 40 and the common reference wire 10 normally form a short circuit and the current setpoint cannot be reached, which allows the controller 2 to verify the reliability of the spare wire 40. Preferably, the voltage measurement is performed while the remote units are switched off.

### NUMERICAL REFERENCES

1—Stimulation System//2—Controller//3—Remote Unit//7—Load Balancing Module//10—Common Reference Wire//12—Digital Controller//13—Remote Unit Digital//20—Wire of Energy//21—Module//22—Energy Recovery Module//22R—Energy Recovery Spare module//30—Data Wire//31—Data Emission/Reception Module//31R—Data Emission/Reception Spare module//40—Spare wire//41—Switch Normally Closed//50—Electrode//60—IC Medium Contact//70—Buffer capacitor//71/72/73/74—Controlled Switches.

1-10. (canceled)

11. A stimulation system adapted to be implanted in a person comprising:
distributed elements comprising:
a controller comprising an energy emission module and a data emission/reception module; and
at least one remote unit comprising at least one energy recovery module and at least one data emission/reception module;
a wired bus connecting all the distributed elements, said bus comprising:
a common reference wire connecting all the distributed elements;
an energy wire connected via a coupling capacitor to the energy emitting module of the controller; and connected directly to the energy recovery modules of the at least one remote unit; and
a data wire connected via a coupling capacitor to each of the data emission/reception modules of all the distributed elements.

12. The stimulation system according to claim 11, wherein the at least one remote unit is selected from stimulation units, measurement units and mixed stimulation and measurement units.

13. The stimulation system according to claim 12, comprising at least two stimulation units and at least one measurement unit.

14. The stimulation system according to claim 11, wherein at least one remote unit further comprises at least one electrode configured to be in contact with the intra-body medium and to measure an electrophysiological signal.

15. The stimulation system according to claim 11, wherein each energy recovery module is assisted by a load balancing module comprising a buffer capacitor.

16. The stimulation system according to claim 11, wherein the wired bus comprises a spare wire to:
transmit event-related data; and/or
be substituted with the common reference wire, the energy wire or the data wire when one of these wires fails.

17. The stimulation system according to claim 16, wherein each of the at least one remote unit comprises a normally closed switch between the common reference wire and the spare wire.

18. A method of measuring physiological signals by a stimulation system according to claim 14, comprising:
connecting to the common reference wire an electrode of the remote unit in contact with the intra-body medium; and
measuring by the controller a voltage between the common reference wire and an IC medium contact of the controller.

19. A method of measuring contact impedance of an electrode of a stimulation system according to claim 14 comprising:
connecting to the common reference wire an electrode of a remote unit in contact with the intra-body medium;
injecting by an intra-body medium contact of the controller a current in the intra-body medium; and
measuring by the controller the voltage between the common reference wire and the IC medium contact of the controller.

20. A method for detecting defects of a stimulation system according to claim 16, comprising:
injecting by the controller a current into the energy wire or the data wire or the spare wire; and
measuring by the controller the voltage between the common reference wire and the wire into which the current has been injected.

21. The stimulation system according to claim 14, wherein the wired bus comprises a spare wire to:
transmit event-related data; and/or
be substituted with the common reference wire, the energy wire or the data wire when one of these wires fails.

22. The stimulation system according to claim 21, wherein each of the at least one remote unit comprises a normally closed switch between the common reference wire and the spare wire.
